(19) United States
(12) Patent Application Publication
(10) Pub. No.: US 2012/0107256 A1
(43) Pub. Date: May 3, 2012

(54) Personal Care Emulsions Comprising Waxy Materials And Organopolysiloxanes

(76) Inventors: Cindy Delvalle, Uccle (BE); Sophie Hanssens, Chastre (BE); Houria Seghir, Nivelles (BE); Sylvie Bouzeloc, Montigny-le Tilleul (BE)

(21) Appl. No.: 13/381,199

(22) PCT Filed: Jun. 28, 2010
(86) PCT No.: PCT/EP10/59135

§ 371 Date: Dec. 28, 2011

(30) Foreign Application Priority Data
Jul. 2, 2009 (GB) GB0911488.5
Jun. 28, 2010 (EP) PCT/EP2010/059135

(51) Int. Cl. A61K 8/92; A61Q 17/04; A61Q 5/06; A61Q 19/08; A61Q 19/10; A61Q 5/12; A61Q 5/02

(52) U.S. Cl. 424/59; 424/78.03; 510/417; 424/65; 514/63; 424/70.12

(57) ABSTRACT

The invention provides an aqueous emulsion for use in a personal care, health care or household care composition. This emulsion comprises at least 1% by weight of a waxy material having a melting point of at least 10.5° C. and at least 1% by weight of an organopolysiloxane which is not miscible with the waxy material. The emulsion also contains an oil having a melting point below 10° C., the oil being miscible with the waxy material when melted and being present in a weight ratio of oil to waxy material in the range 5:95 to 95:5. This permits to obtain stable emulsion at low temperature.

This invention relates to personal care compositions, such as skin care and hair care compositions, cosmetics and toiletries, which contain waxy materials. Waxy materials, particularly vegetable butters, are increasingly popular in personal care compositions. Vegetable butters are plant-derived lipids whose main constituents (generally over 90%) are triglycerides. They are regarded as multifunctional ingredients and are used as emollients, moisturizers, emulsifiers or lubricants. In particular the invention relates to personal care compositions containing organopolysiloxanes (silicones) as well as waxy materials.

Vegetable butters can cause difficulties for the formulator of cosmetic and other personal care compositions due to their crystallization behaviour especially if used at high concentration in emulsion. Crystallization may happen when the temperature drops below the melting point of the butter. Some of these butters have melting points which are close to room temperature or a bit higher. The crystallization tendency of a given butter is primarily dependant on its chemical structure, its concentration, its melting point and its polymorphism properties. In the cosmetic industry the preservation of the consistency and texture of a cosmetic product during its shelf life is important. These properties are associated with the quality of the finished product. Butters used in these applications have a tendency to promote formation of lipid crystal networks. This destabilizes emulsions. This latter phenomenon results in changes in consistency (from creamy to grainy with a rough aspect) and in appearance (visible separated phases of oil or fat can be seen, resulted from droplets coalescence). There are also modifications in rheological properties of the emulsion. Organopolysiloxanes are valuable ingredients of many personal care, health care and household care emulsions, but are generally not miscible with vegetable butters and do not stabilize emulsions containing vegetable butters against destabilization at low temperatures.

JP 2009/019023 describes a hair-dressing emulsified cosmetic comprising (A) rice bran wax and/or hydrogenated rice bran wax, (B) candelilla wax, (C) a non-ionic surfactant, and (D) a branched fatty acid and/or a branched fatty acid ester.

WO 03/013447 describes hair and skin care compositions comprising a silicone fluid, a silicone immiscible substance, and a siloxane-based polyamide.

The article ‘Particle-stabilized emulsions comprised of solid droplets’ by J. Giermanska-Kahn et al in Langmuir 2005, 21, pp 4316-4323 describes kinetically stabilized oil-in-water emulsions comprising paraffin wax crystals by absorbing solid silica particles of colloidal size at the oil/water interface. The resulting emulsion however has a large particle size due to the presence of the silica particles and although stabilized against crystallization on cooling below the melting point of the wax is sensitive to external surfactants. These sorts of emulsions (stabilization by particles) are not appropriate for cosmetics and toiletries formulations.

The article ‘Thermally induced gelling of oil-in-water emulsions comprising partially crystallized droplets: the impact of interfacial crystals’ by F. Thivilliers et al in Langmuir 2008, 24, pp 13364-13375, describes the use of a mixture of protein and low molecular weight surfactant to stabilize an emulsion comprising a butter having a tendency to crystallize. The use of proteins has several disadvantages in a cosmetic product which is intended to have a long shelf life. In general proteins are not compatible with conventional surfactants usually used in this application. Moreover proteins promote bacterial growth and are harder to preserve.

According to one aspect of the invention, a method of stabilizing an aqueous emulsion comprising at least 1% by weight of a waxy material having a melting point of at least 10.5° C., preferably of at least 20° C. and at least 1% by weight of an organopolysiloxane which is not miscible with the waxy material, the method comprises incorporating an oil, which has a melting point below 10° C. and is miscible with the melted waxy material, in the emulsion at a weight ratio of oil to waxy material in the range 5:95 to 95:5. The oil is considered as miscible with the waxy material if the oil can be mixed in the waxy material without phase separation when the waxy material is melted.

Stability at low temperature can be observed by the particle size, which should remain substantially unchanged, and by evaluating the emulsion under the microscope after the emulsion has been submitted to low temperature. The emulsion is stable at low temperature when the particle size of the emulsion does not substantially increase and no crystals are visible under the microscope after the emulsion has been submitted to low temperature.

An aqueous emulsion according to the invention for use in a personal care, health care or household care composition comprises at least 1%, preferably at least 5% by weight of a waxy material having a melting point of at least 10.5° C., preferably at least 1%, preferably at least 5% by weight of an organopolysiloxane which is not miscible with the waxy material, and also contains an oil having a melting point below 10° C., the oil being miscible with the waxy material when melted and being present in a weight ratio of oil to waxy material in the range 5:95 to 95:5.

The invention includes the use of an oil which has a melting point below 10° C. to stabilize at low temperatures an aqueous emulsion for use in a personal care, health care or household care composition, said emulsion comprising at least 1%, preferably at least 5% by weight of a waxy material having a melting point of at least 10.5° C.

Depending on the targeted low temperature at which stability is required, different liquid oils having different melting points can be used. An oil having a melting point of 10° C. may stabilize the emulsion against destabilization at temperatures down to 20° C. or 15° C. An oil having a melting point of 0° C. or below will stabilize the emulsion against destabilization at temperatures down to 5° C. We have found that the addition of an oil of melting point below −20° C. to the vegetable butter or other waxy material forms an emulsion with significant stability improvement at low temperatures of 10° C. and below, especially at very low temperatures of 5° C. down to −10° C. or −20° C.

The waxy material having a melting point of at least 10.5° C., preferably at least 20° C., more preferably at least 25° C. is in many formulations a triglyceride wax derived from plant seeds, fruits, nuts or kernel such as a vegetable butter, but can alternatively be a hydrocarbon wax. A waxy material is preferably a material which is plastic or malleable at temperatures of 15-20° C., has a melting point of at least 10.5° C., and has a low viscosity when melted.

For the purpose of the current invention vegetable butters are defined by having a titer or melting point of at least 10.5° C. Some vegetable butters have a melting point or titer of below 40.5° C. (or 45° C.) but above 20° C. (“Oil of nature” by J. O'Lenick according to AOCS method Tr 1a-64T). Examples of vegetable butters which can be used in the emulsions of the invention include those frequently used in skin care and other personal care and cosmetic applications such as mango butter, shea butter, cocoa butter, and kokum butter, which generally have melting points in the range 30-45° C. Further examples include illipe, cupuacu, murumuru, sal, tucuma and mowrah butter. Some vegetable triglyceride products have the properties of vegetable butters but are commonly called oils, for example coconut oil which is frequently used in skin care and other personal care and cosmetic applications and has a melting point in the range 20-28° C., mango kernel oil, of melting point 34-43° C., palm oil of melting point 37° C., palm olein and palm stearin. Such products can be used as the waxy material in this invention. Mixtures of butter can be used to produce this emulsion. There are some man-made butters being introduced to the cosmetic industry which create an aesthetic match of naturally occurring butters. These butters tend to be refined cosmetic vegetable oils such as olive, avocado, macadamia, jojoba and almond that have either undergone hydrogenation, or have been physically blended with hydrogenated or fractionated vegetable oils. Such man-made butters can be used separately or in combination with other vegetable butters as the waxy material in the emulsions of the invention. The emulsion of the invention preferably contains at least 1% by weight vegetable butter, more preferably at least 5 or 10% vegetable butter, and may contain up to 50 or 70% by weight vegetable butter.

Other waxy materials derived from plant seeds, fruits, nuts or kernel which can be used in the emulsions of the invention include palm wax, rice bran wax or soy wax. Other waxes comprising carboxylic esters, particularly triglycerides, which can be used include beeswax, lanolin, tallow, carnauba, candelilla and tribehenin.

The waxy material can alternatively be a hydrocarbon wax such as a petroleum-derived wax, particularly a paraffin wax or microcrystalline wax, a Fischer-Tropsch wax, ceresin wax, a polyethylene wax or a mixture thereof. Paraffin waxes contain predominantly straight-chain hydrocarbons with an average chain length of 20 to 30 carbon atoms. Microcrystalline wax contains a higher percentage of branched hydrocarbons and naphthenic hydrocarbons. Other organic hydrocarbon waxes that can be used are montan wax (also known as lignite-wax), ozokerite or slag wax.

The waxy material can alternatively be a long chain fatty acid, a long chain fatty alcohol, a long chain fatty amine, a long chain fatty amide, an ethoxylated fatty acid or fatty alcohol, or a long chain alkyl phenol. In general the long chain of the fatty acid, alcohol, amine or amide is an alkyl group of at least 12 and preferably at least 16 carbon atoms, often up to 30 or more carbon atoms. The waxy material can alternatively be a polyether wax, for example a solid polyether polyol or a waxy polyvinyl ether, or can be a silicone wax, generally a polysiloxane containing hydrocarbon substituents having 12 or more carbon atoms.

The waxy material having a melting point of at least 10.5° C.—is present at a concentration of at least 1% by weight in the emulsion. The crystallization behaviour which this invention ameliorates occurs especially if the waxy material is used at high concentration in emulsion, for example at least 10 or 15% by weight in the emulsion up to a concentration of 50 or even 70% by weight in the emulsion.

The organopolysiloxane generally contains siloxane units independently selected from (R₃SiO_{0.5}), (R₂SiO), (RSiO₁₅), or (SiO₂) siloxane units, commonly referred to as M, D, T, and Q siloxane units respectively, where R may be any organic group containing 1-30 carbon atoms, for example an alkyl group containing 1 to 6 carbon atoms, particularly methyl or ethyl, a phenyl group, an aralkyl group or a substituted alkyl group containing 1 to 6 carbon atoms such as an aminoalkyl group or a quaternised aminoalkyl group. Suitable organopolysiloxanes include linear or branched polydiorganosiloxanes consisting wholly or mainly of D units, for example polydimethylsiloxanes, or functionally substituted polydimethylsiloxanes in which some of the methyl groups are replaced by substituted alkyl groups, or branched siloxane resins containing T and/or Q units, for example DT resins containing D and T units or MQ resins containing M and Q units. Polydiorganosiloxanes are widely used in hair care compositions as conditioners, for example polydimethylsiloxanes or substituted polydimethylsiloxanes in which some of the methyl groups are replaced by aminoalkyl groups. The organopolysiloxane may include the very high molecular weight polyorganosiloxanes known as silicone gums.

The organopolysiloxane is present at a concentration of at least 1% by weight in the emulsion, preferably at least 5%, and may for example be present at a concentration of from 10 or 15% by weight up to 50 or 70% by weight.

The oil having a melting point below 10° C. or other targeted low temperature at which stability is required can for example be a hydrocarbon oil such as a mineral oil or an ester oil such as a triglyceride oil. A mineral hydrocarbon oil can for example be a petroleum fraction or can be formed by chemical reaction such as hydrogenation, for example hydrogenated polydecene. Triglyceride oils having the required low melting point usually contain a high proportion of unsaturated and polyunsaturated fatty acid residues, particularly if the targeted low temperature is below 10° C., for example below 0° C. or below −10° C. or −20° C. Examples include shea oil, soybean oil and jojoba oil, sunflower oil, grape seed oil, rapeseed oil, sunflower oil, maize oil, olive oil, evening primrose oil, borage oil, flax seed oil, rice bran oil, castor oil and linseed oil, all of which have melting points well below 0° C. and are suitable for enhancing stability at targeted low temperatures below 0° C. Further examples of triglyceride oils include cottonseed oil, groundnut (peanut) oil, sesame oil and tung oil, all of which have melting points close to 0° C. and are suitable for enhancing stability at targeted temperatures of 10 or 15° C. Mixtures of liquid oils, for example mixtures of hydrocarbon oil and triglyceride oil or mixtures of different triglyceride oils, can be used to stabilize the emulsion at low temperature.

The oil having a melting point below 10° C. or other targeted low temperature can alternatively be an organomodified silicone oil which is miscible with the melted vegetable butter or other melted waxy material, for example a polyphenylmethylsiloxane such as the oil sold by Dow Corning under the trade mark DC 556.

The oil having a melting point below 10° C. or other targeted low temperature can alternatively be a cyclic organopolysiloxane such as decamethylcyclopentasiloxane or octamethylcyclotetrasiloxane, or another low molecular weight silicone which is miscible with the melted vegetable butter or other waxy material. We have found that such cyclic organopolysiloxanes, and linear polyorganosiloxanes of similar molecular weight, are miscible with vegetable butters such as shea butter and mango butter.

Solvents can be used if necessary in combination with the liquid oils to improve low temperature stability. One example of such a solvent is ethanol.

The emulsions of the invention preferably contain at least one surfactant suitable to emulsify the waxy material and the organopolysiloxane as an oil-in water or water-in-oil emulsion. The surfactant can be any of those known for use in personal care products and can be selected from anionic, cationic, non-ionic, amphoteric and polymeric surfactants. More than one surfactant can be used, for example different types of surfactants or more than one surfactant of the same type (ionic or non-ionic).

Examples of non-ionic surfactants include polyoxyalkylene alkyl ethers, for example polyethylene glycol long chain (12-20° C.) alkyl ethers such as Steareth-21 (Brij 721 from Uniqema) and Ceteth-20 (Brij 58 from Uniqema), polyoxyalkylene sorbitan ethers, polyoxyalkylene alkoxylate esters, polyoxyalkylene alkylphenol ethers, ethylene glycol propylene glycol copolymers, long chain fatty acid amides and their derivatives such as cocoamide diethanolamide (Cocoamide DEA), and alkylpolysaccharides.

Examples of suitable anionic surfactants include sodium ethoxylated lauryl sulfate (sodium laureth sulfate or SLES), sodium lauryl sulphate, sodium alkylbenzenesulfonate, sodium xylenesulfonate, ammonium laureth sulfate, sodium polynaphthalenesulfonate, ammonium lauryl sulfate, and ammonium xylenesulfonate.

Examples of suitable cationic surfactants include quaternary ammonium halides such as octyl trimethyl ammonium chloride, dodecyl trimethyl ammonium chloride, hexadecyl trimethyl ammonium chloride, octyl dimethyl benzyl ammonium chloride, decyl dimethyl benzyl ammonium chloride, didodecyl dimethyl ammonium chloride, dioctadecyl dimethyl ammonium chloride, tallow trimethyl ammonium chloride and coco trimethyl ammonium chloride as well as corresponding hydroxides or other salts of these materials, fatty amines and basic pyridinium compounds, quaternary ammonium bases of benzimidazolines and polypropanolpolyethanol amines.

Examples of suitable amphoteric surfactants include cocamidopropyl betaine (CAPB), cocamidopropyl hydroxysulfate, cocobetaine, sodium cocoamidoacetate, cocodimethyl betaine, N-coco-3-aminobutyric acid and imidazolinium carboxyl compounds.

Examples of suitable polymeric surfactants include polyvinyl alcohol, proteins, ethylene oxide/propylene oxide block copolymer surfactants (commercially available under the trade name Pluronic), and polyether/polyester copolymers (available under the trade names Marloquest HSCB and Marloquest UK).

The water content of the aqueous emulsion is usually at least 10% by weight and may be up to 85 or 90% by weight. The water content of a skin cream emulsion may typically be in the range 15 to 50% by weight, while a shower gel or hair shampoo will have a higher water content.

The surfactant content of the emulsion is usually at least 1% by weight and can for example be in the range 2 to 10% for a skin cream such as a moisturiser or can be substantially higher, for example up to 25 or 40% for a cleaning product such as a shampoo.

The emulsions can be prepared by various processes. One process consists in producing separately two emulsions. One emulsion will be an emulsion of the organopolysiloxane only and the second an emulsion of the waxy material such as vegetable butter. Usually the vegetable butter and the low melting point oil are mixed to form a butter/oil blend before emulsifying. The butter emulsion and silicone emulsion separately prepared are then mixed. Alternatively the emulsion can be prepared by mixing the waxy material, organopolysiloxane and low melting point oil and emulsifying them together.

Because organopolysiloxanes (silicones) are highly hydrophobic, stable emulsions can be difficult to produce mechanically. To overcome this, the silicone can be mixed with a surfactant and a small amount of water under high mechanical shear to form a non-Newtonian “thick phase” emulsion, which has a very high viscosity at low shear rates (much more viscous at low shear rate than the silicone polymer alone) and often exhibits a yield stress (viscoplastic behaviour). The surfactant content of the “thick phase” can for example be in the range 2% up to 10 or 20% by weight, with the amount of water being at least 0.5%, preferably at least 1% up to 10 or 20%. The resulting emulsion can be diluted with further water and surfactant. This “thick phase” process can be used to prepare the emulsion of a mixture of the silicone with the waxy material and the oil. Alternatively the oil can be mixed with surfactant solution and emulsified employing a high shear or a high pressure device. Examples of high shear devices are Ultraturax (IKA Gmbh), Rannie (APV), or sonolator (Sonic) respectively.

The emulsions of the invention are useful in personal care applications such as on hair, skin, mucous membrane or teeth. In skin care applications, the silicone is lubricious and will improve the properties of skin creams, skin care lotions, moisturisers, facial treatments such as acne or wrinkle removers, personal and facial cleansers such as shower gels, liquid soap, hand sanitizers, bath oils, perfumes, fragrances, colognes, sachets, deodorants, sun protection creams, lotions and wipes, colour cosmetics such as foundations and mascaras, self tanning creams and lotions, pre-shave and after shave lotions, after sun lotion and creams, antiperspirant sticks, soft solid and roll-ons, shaving soaps and shaving lathers. The vegetable butter or similar waxy material will melt readily on contact with the skin giving an aesthetically pleasing soothing effect and can also bring benefits to personal care products ranging from oxidative stability, humectant properties, anti-inflammatory properties. The emulsions of the invention can likewise be used in hair care products such as shampoos, rinse-off and leave-on hair conditioners, hair styling aids, such as sprays, mousses and gels, hair colorants, hair relaxers, permanents, depilatories, and cuticle coats, where the silicone for example provides styling and conditioning benefits. In cosmetics, both the silicone and the vegetable butter function as a levelling and spreading agent for pigment in make-ups, colour cosmetics, compact gel, cream and liquid foundations (water-in-oil and oil-in-water emulsions, or anhydrous lotions), blushes, eye liners, eye shadows, mascaras, and make up removers. The emulsion of silicone and waxy material is likewise useful as a delivery system for oil and water soluble substances such as vitamins, fragrances, emollients, colorants, organic sunscreens, or pharmaceuticals.

The invention is illustrated by the following Examples, in which parts and percentages are by weight.

### EXAMPLE 1

18 g of melted HY-4003 Shea Butter (from Dow Corning) and 18 g Shea oil ultra refined (from Biochemica) were weighed and blended in a bottle. 36 g trimethylsilyl-terminated polydimethylsiloxane 200 fluid (PDMS) of viscosity 350 centiStokes was weighed separately.

1.23 g melted Brij 721 (from Uniqema), 2.47 g melted Brij 58 (from Uniqema), 2.3 g warm water, 2 g PDMS and 2 g of Shea butter/Shea oil liquidified blend were weighed together in a dental pot. The overall mixture was emulsified using a Hauschild Dental mixer (DAC 40) for 36 seconds at 2750 RPM. The Shea butter/Shea oil blend and silicone were added in successive portions of 2 g melted butter/oil blend and 2 g PDMS until all the butter/oil blend and all the silicone were incorporated in the blend. Every addition is followed by a mix in the Dental mixer, and with every second addition of butter/oil blend and silicone 2.3 g water was added to lower the viscosity of the mixture. At the end the remaining water was added in several fractions (23.32 g in total). Each water addition is followed by a mix in the Dental mixer. Then potassium sorbate and phenoxyethanol biocides and ethylenediaminetetraacetic acid disodium salt (EDTA-2NA) were added to give the formulation shown in Table 1 below.

The effectiveness of any candidate liquid oil can be tested by mixing equal amounts of melted vegetable butter and same amount of liquid oil and emulsifying this blend with emulsifying agents, water and silicone using the process described above. The resulting emulsion can be subjected to a temperature profile depending on the targeted low temperature and the particle sizes are measured subsequently. If the particle size after submitting the emulsion at low temperature is unchanged, the oil is deemed to be effective at stabilizing butter/silicone emulsion.

### EXAMPLE 2

Example 1 was repeated using 27 g Shea butter and 9 g Shea oil as the butter/oil blend.

### COMPARATIVE EXAMPLE C1

Example 1 was repeated using 36 g Shea butter in place of the butter/oil blend.

TABLE 1

| | Weight % | | |
|---|---|---|---|
| | Comparative | Weight % | Weight % |
| | Example Cl | Example 1 | Example 2 |
| PDMS | 36.00 | 36.00 | 36.00 |
| Water | 23.32 | 23.32 | 23.32 |
| Potassium Sorbate | 0.04 | 0.04 | 0.04 |
| Phenoxyethanol | 0.90 | 0.90 | 0.90 |
| EDTA-2 NA | 0.04 | 0.04 | 0.04 |
| Brij 721 | 1.23 | 1.23 | 1.23 |
| Brij 58 | 2.47 | 2.47 | 2.47 |
| Shea butter | 36.00 | 18.00 | 27.00 |
| Shea oil | — | 18.00 | 9.00 |

In each of Examples 1, 2 and C1 a water-in-oil emulsion was produced which was stable at room temperature (RT). The particle size of the emulsions was measured with a Malvern Mastersizer 2000 and is indicated in Table 2.

Each emulsion was tested under various cycles of ageing in a climatic chamber (supplied by Vötsch Industrietechnik GmbH; Equipment type: VT 4011). The cycles of ageing were:

24 h at 10° C.

24 h at 5° C.

24 h at 0° C.

24 h at −5° C.

24 h at −10° C.

24 h at −18° C.

After each cycle of ageing the emulsions were left to go back to room temperature. They were subsequently checked for visual appearance and emulsion particle size. If a sample had acceptable appearance the sample was placed back in the climatic chamber for the next cycle at a lower temperature and the testing was repeated until there was a visible change when the sample was allowed to warm to room temperature, for example a grainy appearance or visible oil leaching. The particle size of each emulsion was measured by the Malvern Mastersizer 2000 after each cycle of ageing and is recorded in Table 2 below.

The emulsion of Comparative Example C1 containing only shea butter (no liquid oil) was stable at 15° C. but after 24 hours at 10° C. the emulsion displayed graininess. At this point particle size could not be determined with accuracy by the Malvern Mastersizer 2000, but it could be confirmed by optical microscopy (Zeiss Axioplan objective 100× operating in transmitted illumination) that large lumps of Shea butter are visible.

The emulsion of Example 1 containing 50:50 shea butter and shea oil was subjected to 24 hour cycles in the climatic chamber repeated down to −10° C. and the emulsion did not feature any signs of destabilization. Confirmation of the stability has been seen with a constant particle size down to −10° C., as shown in Table 2. No change in aspect was observed after 24 h at −10° C.

The emulsion of Example 2 containing a 75:25 blend of Shea butter and shea oil was stable down to −5° C. This emulsion displayed a destabilization at −10° C. After 24 hours at −10° C. the emulsion demonstrated grains and no particle size could be measured by the Malvern Mastersizer 2000. Optical microscopy revealed shea lumps which is a sign of destabilization of the emulsion. The emulsion of Example 2 thus showed substantially improved low temperature storage properties than the emulsion of Comparative Example C1, although it was not so stable at −10° C. as the emulsion of Example 1.

TABLE 2

| | | Particle Size (μm) | | |
|---|---|---|---|---|
| | | Dv(0, 1) | Dv(0, 5) | Dv(0, 9) |
| Comparative | after preparation | 0.133 | 0.426 | 1.709 |
| Example C1 | (RT) | | | |
| | After 24 h at 10° C. | n/a | n/a | n/a |
| Example 2 | after preparation | 0.127 | 0.415 | 1.838 |
| | (RT) | | | |
| | After 24 h at 5° C. | 0.113 | 0.314 | 1.281 |
| | After 24 h at 0° C. | 0.11 | 0.323 | 1.406 |
| | After 24 h at −5° C. | 0.107 | 0.313 | 1.339 |
| | After 24 h at −10° C. | n/a | n/a | n/a |
| Example 1 | after preparation | 0.093 | 0.255 | 1.616 |
| | (RT) | | | |
| | After 24 h at 5° C. | 0.126 | 0.425 | 2.144 |
| | After 24 h at 0° C. | 0.114 | 0.388 | 2.069 |
| | After 24 h at −5° C. | 0.116 | 0.397 | 2.116 |
| | After 24 h at −10° C. | 0.119 | 0.396 | 2.045 |
| | After 24 h at −18° C. | n/a | n/a | n/a |

n/a: not applicable

### EXAMPLE 3

Example 1 was repeated using mango butter available from Dow Corning under the trade name HY-4001 in place of the shea butter and a vegetable oil available from Dow Corning under the trade name HY-4008 in place of the shea oil. The same process and level of active materials were used as in Example 1.

### COMPARATIVE EXAMPLE C2

A comparative example C2 was carried out which repeated comparative example C1 but using mango butter HY-4001 in place of the shea butter. Examples 3 and C2 were tested as described for Example 1 and the results are reported in Table 3.

TABLE 3

| | | Particle Size (μm) | | |
|---|---|---|---|---|
| | | Dv(0, 1) | Dv(0, 5) | Dv(0, 9) |
| Comparative | after preparation | 0.121 | 0.421 | 3.028 |
| Example C2 | (RT) | | | |
| | After 24 h at 10° C. | n/a | n/a | n/a |
| Example 3 | after preparation | 0.089 | 0.235 | 1.356 |
| | (RT) | | | |
| | After 24 h at 5° C. | 0.109 | 0.343 | 1.758 |
| | After 24 h at 0° C. | 0.101 | 0.329 | 1.751 |
| | After 24 h at −5° C. | 0.114 | 0.374 | 1.84 |
| | After 24 h at −10° C. | 0.118 | 0.386 | 1.866 |
| | After 24 h at −18° C. | n/a | n/a | n/a |

The emulsion of Comparative Example C2 containing only mango butter (no liquid oil) was stable at room temperature but after 24 hours at 10° C. the emulsion displayed graininess.

The emulsion of Example 3 containing equal weights of mango butter and vegetable oil was subjected to 24 hour cycles in the climatic chamber repeated down to −10° C. and the emulsion did not feature any signs of destabilization. Confirmation of the stability has been seen with a constant particle size down to −10° C., as shown in Table 3.

### EXAMPLE 4

Emulac (Milk casein) was obtained from Brenntag, Shea butter HY-4003 was from Dow Corning and grape seed oil was purchased from ID BIO SAS. The shea butter was melted in a microwave oven until no crystals are observed. The melted shea butter was then mixed with grape seed oil and trimethylsilyl-terminated polydimethylsiloxane 200 fluid (PDMS) of viscosity 100 centiStokes. No re-crystallization happened.

The casein protein was dissolved in water at a concentration of 6% using a simple propeller. Before dissolving the protein Neolone DSP biocide has been added to water in a concentration of 1%. An emulsion of 60% oil phase was prepared by high shear mixing (Ultraturrax, 1 minute, max speed) followed by a refinement employing a Rannie homogenizer operating at 720 bars. The formulation of the emulsion is shown in Table 4 below.

### COMPARATIVE EXAMPLE C3

In a comparative example C3, an emulsion was prepared using the process of Example 4 but with the grapeseed oil replaced by further shea butter. The formulation of this emulsion is given in Table 4.

### EXAMPLE 5

An emulsion containing a higher concentration of shea butter and grape seed oil with no PDMS was prepared using the process of Example 4 and the formulation is shown in Table 4.

TABLE 4

| | Weight % | | |
|---|---|---|---|
| | Comparative | Weight % | Weight % |
| | Example C3 | Example 4 | Example 5 |
| PDMS 200 fluid 100 cst | 30.00 | 30.00 | — |
| Water | 37.20 | 37.20 | 37.20 |
| Neolone DSP | 0.4 | 0.4 | 0.4 |
| Protein | 2.40 | 2.40 | 2.40 |
| Shea butter | 30.00 | 7.50 | 15.00 |
| Grape seed oil | — | 22.50 | 45.00 |

The emulsions of Examples 4 and 5 and comparative example C3 were tested as described for Example 1 and the results are reported in Table 5.

TABLE 5

| | | Particle Size (μm) | | |
|---|---|---|---|---|
| | | Dv(0, 1) | Dv(0, 5) | Dv(0, 9) |
| Comparative | after preparation | 0.177 | 0.573 | 1.268 |
| Example C3 | (RT) | | | |
| | After 24 h at 10° C. | 0.098 | 0.421 | 1.179 |
| | After 24 h at 5° C. | 0.111 | 0.438 | 1.145 |
| | After 24 h at 0° C. | 0.111 | 0.447 | 1.193 |
| | After 24 h at −5° C. | 0.169 | 0.526 | 1.253 |
| | After 24 h at −10° C. | n/a | n/a | n/a |
| Example 4 | after preparation | — | — | — |
| | (RT) | | | |
| | After 24 h at 5° C. | 0.189 | 0.469 | 1.004 |
| | After 24 h at 0° C. | 0.182 | 0.465 | 1.062 |
| | After 24 h at −5° C. | 0.156 | 0.432 | 0.972 |
| | After 24 h at −10° C. | 0.147 | 0.54 | 1.316 |
| | After 24 h at −18° C. | 0.141 | 0.568 | 1.568 |
| Example 5 | after preparation | 0.142 | 0.457 | 0.967 |
| | (RT) | | | |
| | After 24 h at 5° C. | 0.209 | 0.505 | 1.019 |
| | After 24 h at 0° C. | 0.226 | 0.528 | 1.128 |
| | After 24 h at −5° C. | 0.2 | 0.499 | 0.990 |
| | After 24 h at −10° C. | 0.187 | 0.671 | 1.473 |
| | After 24 h at −18° C. | 0.164 | 0.751 | 2.134 |

The emulsion of Comparative Example C3 containing shea butter with no liquid oil using milk protein as emulsifier was stable down to −5° C. but after 24 hours at −10° C. the emulsion displayed graininess. The emulsion of Comparative Example C3 was thus considerably more stable at low temperatures than the emulsions of Comparative Examples C1 containing vegetable butter with no liquid oil using polyoxyalkylene alkyl ether surfactants as emulsifier, confirming the teaching of Thivilliers et al in Langmuir 2008, 24, pp 13364-13375 that protein stabilizes an emulsion comprising a butter having a tendency to crystallize.

The emulsions of Examples 4 and 5 remained stable with little change in particle size right down to −18° C. A comparison of Example 4 with comparative example C3 shows that the addition of low melting grape seed oil imparted substantial extra low temperature stability to an emulsion containing vegetable butter even when the emulsion contained protein stabilizer.

1. An aqueous emulsion for use in a personal care, health care or household care compositions, said emulsion comprising at least 1% by weight of a waxy material having a melting point of at least 10.5° C. and at least 1% by weight of an organopolysiloxane which is not miscible with the waxy material, wherein the emulsion also contains an oil having a melting point below 10° C., the oil being miscible with the waxy material when melted and being present in a weight ratio of oil to waxy material in the range 5:95 to 95:5.

2. The emulsion according to claim 1, wherein the waxy material is a vegetable butter.

3. The emulsion according to claim 2, wherein the vegetable butter is shea butter or mango butter or cocoa butter or kokum butter.

4. The emulsion according to claim 2, wherein the emulsion contains 1 to 70% by weight vegetable butter.

5. The emulsion according to claim 1, wherein the waxy material is a hydrocarbon wax.

6. The emulsion according to claim 1, wherein the oil has a melting point below 10° C.

7. The emulsion according to claim 1, wherein the oil is a liquid vegetable oil or a mixture comprising at least one liquid vegetable oil.

8. The emulsion according to any of claim 1, wherein the oil is a hydrocarbon oil.

9. The emulsion according to claim 1, wherein the oil is a silicone-modified oil or a low molecular weight silicone which is miscible with the waxy material.

10. The emulsion according to claim 1, wherein the weight ratio of oil to waxy material is in the range 10:90 to 90:10.

11. The emulsion according to claim 10, wherein the weight ratio of oil to waxy material is in the range 50:50 to 75:25.

12. The emulsion according to any of claim 1, wherein the emulsion contains 10 to 60% by weight of the organopolysiloxane which is not miscible with the waxy material.

13. The emulsion according to any of claim 1, wherein the emulsion contains 10 to 85% by weight water.

14. A personal care composition which is a skin care, hair care or cosmetic composition in aqueous emulsion form, wherein the aqueous emulsion comprises at least 1% by weight of a waxy material having a melting point of at least 10.5° C. and at least 1% by weight of an organopolysiloxane which is not miscible with the waxy material, and the emulsion also contains an oil having a melting point below 10° C., the oil being miscible with the melted waxy material and being present in a weight ratio of oil to waxy material in the range 5:95 to 95:5.

15. A method of stabilizing an aqueous emulsion comprising at least 5% by weight of a waxy material having a melting point of at least 10.5° C. and at least 1% by weight organopolysiloxane which is not miscible with the melted waxy material, against destabilization at low temperatures, wherein an oil, which has a melting point below 10° C. and is miscible with the waxy material when melted is incorporated in the emulsion at a weight ratio of oil to waxy material in the range 5:95 to 95:5.

16. (canceled)
